(19) United States
(12) Patent Application Publication
(10) Pub. No.: US 2008/0113891 A1
(43) Pub. Date: May 15, 2008

(54) Underarm cleanser

(76) Inventors: Anahid Arsharuni, Tujunga, CA (US); Morton A. Westman, Oak Brook, IL (US)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 11/984,066

(22) Filed: Nov. 13, 2007

Related U.S. Application Data
(60) Provisional application No. 60/858,367, filed on Nov. 13, 2006.

(51) Int. Cl. A61K 8/34; A61Q 19/10

(52) U.S. Cl. 510/137

(57) ABSTRACT

A cleanser composition for removal of antiperspirant residue that includes insoluble metal salts comprises a surfactant, 0.01 to 2.5 wt % of chelating agent, and a diluent.

### CROSS-REFERENCE TO RELATED APPLICATION

This application is based on U.S. Provisional Application No. 60/858,367, filed Nov. 13, 2006, the priority of which is hereby claimed.

### BACKGROUND OF THE INVENTION

The present invention relates to body cleansers (body washes).

Typical body washes and cleansers are used to remove sebum, dirt and various residues from a user's skin when, for example, taking a shower. However, although known washes and cleansers are effective for general cleaning, they are not effective in removing underarm residues primarily consisting of antiperspirant residue and including metallic antiperspirant agents (primarily aluminum salts). Such residues form in the arm pit area, leading to a feeling of dirtiness in that area that persists through current cleansing techniques. Such residues create the tactile sensation of drag or friction when the hand is passed through the underarm during cleansing and may leave the user feeling unclean and discontent with their cleansing regimen. Such residues also discolor (primarily yellow) garments that come into direct contact with these areas, even after being freshly cleaned.

The present inventors have discovered a cleansing composition which is effective in removing underarm residues primarily consisting of antiperspirant residue and including metallic residues of antiperspirants from a user's skin, and in particular in their underarm areas.

### SUMMARY OF THE INVENTION

According to this invention, a chelating agent is combined with cleaning surfactants so as to provide a composition which will remove residues primarily consisting of antiperspirant residue and including insoluble antiperspirant metal salts from a user's skin, and in particular from their underarm areas.

Although chelating agents have been previously used in personal care products for various purposes for example in hair dye formulations to trap unwanted metals and thus prevent premature oxidation of the dyestuffs therein and distortion of their intended color, and in other products to increase the effectiveness of microbiological preservatives, their use in body cleaners to enhance the cleansing process and to specifically assist the removal of metallic salt is not previously known. Example formulations of the inventive composition are found in Table 1.

TABLE 1

| | | | | FOR- | | | | |
|---|---|---|---|---|---|---|---|---|
| | INGREDIENT - | | INGREDIENT - | MULA | CONC. RANGE | | | |
| | TRADE NAME | SOURCE | INCI NAME | 071806-01 | Broad | Pref. | PURPOSE | ALTERNATES |
| 1 | Deionized Water | Local | Water | 60.880 | | | Diluent, solvent | None |
| 2 | Natrosol 250 | (1) | Hydroxyethyl- | 1.750 | 0.1-5.0 | 0.2-2.0 | Thickening, provide | Numerous including: various |
| | HHR CS | | cellulose | | | | lubricity during | substituted Cellulosics; |
| | | | | | | | appllication | Carbomer |
| 3 | Plantaren 2000 | (2) | Decyl Glucoside | 10.000 | 1.0-40 | 5.0-20 | Mild nonionic | Anionic & nonionic surfactants |
| | (97% active) | | | | | | surfactant for | (but none quite as appropriate) |
| | | | | | | | cleansing & formula | including: Sodium Lauryl |
| | | | | | | | homogeneity | Sulfate; Ammonium Laureth |
| | | | | | | | | Sulfate |
| 4 | Crodasinic LS-30 | (3) | Sodium Lauroyl | 20.000 | 1.0-60 | 5.0-30 | Mild anionic | Numerous anionic surfactants |
| | (30% active) | | Sarcosinate | | | | surfactant for | (but none quite as appropriate) |
| | | | | | | | cleansing & formula | including: TEA Lauryl Sulfate; |
| | | | | | | | homogeneity | Ammonium Cocoyl Isethionate |
| 5 | Monamid CMA | (4) | Cocamide MEA | 3.000 | 0.5-20 | 2.0-10 | Lather thickener; skin | Numerous amides and amine |
| | | | | | | | conditioner | oxides including: PPG-1 |
| | | | | | | | | Hydroxyethyl Caprylamide; |
| | | | | | | | | Lauramide DEA. |
| 6 | Monateric | (4) | Cocamidopropyl- | 2.000 | 0.5-30 | 2.0-15 | Mild amphoteric | Numerous amphoteric |
| | CAB-LC | | betaine | | | | surfactant for lather | surfactants inclucing: Oleyl |
| | (35% active) | | | | | | thickening & skin | Betaine; Behenyl Betaine; |
| | | | | | | | conditioning | |
| 7 | Stepan EGMS | (5) | Ethylene Glycol | 1.500 | 0.1-6.0 | 0.5-3.0 | Opacifier & pearlizing | Ethylene glycol distearate; latex |
| | | | Monostearate | | | | agent | emulsions |
| 8 | Versene NA | (6) | Disodium EDTA | 0.100 | 0.01-2.5 | 0.05-1.0 | Chelating agent for | Other forms of EDTA |
| | (Disodium | | | | | | removal of metal salts | (Ethylenediamine Tetraacetic |
| | EDTA) | | | | | | & preservation aid | Acid) |
| 9 | Aloe Vera | (7) | Aloe Barbadensis | 0.010 | 0.001-10 | 0.01-3.0 | Natural ingredient for | Numerous including: Althaea |
| | Powder | | Leaf Juice | | | | skin soothing. | Officinalis; Comfrey; Allantoin; |
| | 200:1 Extract | | Powder | | | | | Calendula Officinalis; |
| 10 | Hydroglycolic | (7) | Anthemis Nobilis | 0.010 | 0.001-10 | 0.01-3.0 | | Nasturtium Officinale |
| | Extract of | | Flower Extract | | | | | Extract |
| | Roman Camomile | | | | | | | |
| 11 | Glydant | (8) | DMDM | 0.500 | 0.01-3.0 | 0.2-2.0 | Microbiological | Numerous including: para-bens, |
| | | | Hydantoin | | | | preservative | Imidazolidinyl Urea; |
| | | | | | | | | Methylchloroisothiazolinone |
| 12 | BA 24234 | (9) | Fragrance | 0.250 | 0.05-3.0 | 0.05-2.0 | Fragrance | Numerous |
| | | | | 100.000 | | | | |

Viscosity (25 C., RVT, Sp6; 50 RPM) = approx. 11,200 cps; pH = 7.2 +/− 0.3 (25 C.)
Manufacture: Slowly sprinkle Natrosol to water at RT with rapid mixing. When fully suspended add 3 and 4. Then reduce stirring speed to slow-moderate and Heat to 72-77 C. When free of clumps, fish eyes etc., add 5-8, in order. Allow to cool and add remainder of ingredients below 35° C.
(1) Aqualon Division of Hercules Inc
(2) Cognis Corporation, Care Chemicals
(3) Croda Inc.
(4) Uniqema
(5) Stepan Company
(6) Dow Chemical
(7) International Sourcing Incorporated
(8) Lonza, Inc.
(9) Belle Aire Fragrances

We claim:

1. A cleanser composition which is effective in removing residues primarily consisting of antiperspirant residue and including insoluble antiperspirant metal salts from a user's skin and in particular the underarm area that comprises a surfactant, an effective amount of a chelating agent and a diluent.

2. A cleanser composition according to claim 1, including a non-ionic surfactant, an anionic surfactant, and an amphoteric surfactant.

3. A cleanser composition according to claim 2, wherein said chelating agent comprises disodium ethylenediamine tetracetic acid. (disodium EDTA)

4. A cleanser composition according to claim 3, containing 0.01 to 2.5 wt % disodium EDTA.

5. A cleanser composition according to claim 4, containing 0.05 to 1.0 wt % disodium EDTA.

6. A cleanser composition according to claim 1, wherein said diluent is water.

7. A cleanser composition according to claim 1, including a thickening agent, a skin conditioner, an opacifying agent, and fragrance.

8. A cleanser composition according to claim 8, including a microbiological preservative.
